(19) United States
(12) Patent Application Publication
(10) Pub. No.: US 2013/0253256 A1
(43) Pub. Date: Sep. 26, 2013

(54) APPARATUSES, SYSTEMS, AND METHODS FOR USE AND TRANSPORT OF MAGNETIC MEDICAL DEVICES WITH TRANSPORT FIXTURES OR SAFETY CAGES

(71) Applicants: DAVID B. GRIFFITH, Cincinnati, OH (US); Kevin M. Huey, Cincinnati, OH (US); Christoper J. Hess, Cincinnati, OH (US); Sean P. Conlon, Loveland, OH (US)

(72) Inventors: DAVID B. GRIFFITH, Cincinnati, OH (US); Kevin M. Huey, Cincinnati, OH (US); Christoper J. Hess, Cincinnati, OH (US); Sean P. Conlon, Loveland, OH (US)

(21) Appl. No.: 13/425,047

(22) Filed: Mar. 20, 2012

(51) Int. Cl. A61N 2/10

(52) U.S. Cl. 600/12

(57) ABSTRACT

Various embodiments of spacer apparatuses, and transport and/or storage apparatuses, for use with magnetic platforms configured to be magnetically coupled to a medical device within a body cavity of a patient.

### BACKGROUND

1. Field of the Invention

The present invention relates generally to magnetic medical devices and, more particularly, but not by way of limitation, to apparatuses, systems, and methods for use and transport of magnetic platforms or positioning apparatuses (that are configured to be magnetically coupled to medical devices) with spacers or safety cages, and/or transport fixtures.

2. Description of Related Art

For illustration, the background is described with respect to medical procedures (e.g., surgical procedures), which can include laparoscopy, transmural surgery, and endoluminal surgery, including, for example, natural orifice transluminal endoscopic surgery (NOTES), single-incision laparoscopic surgery (SILS), and single-port laparoscopy (SLP).

Compared with open surgery, laparoscopy can result in significantly less pain, faster convalescence and less morbidity. NOTES, which can be an even less-invasive surgical approach, may achieve similar results. However, issues such as eye-hand dissociation, a two-dimensional field-of-view, instrumentation with limited degrees of freedom, and demanding dexterity requirements can pose challenges for many laparoscopic and endoscopic procedures. One limitation of laparoscopy can be the fixed working envelope surrounding each trocar. As a result, multiple ports may be used to accommodate changes in position of the instruments or laparoscope, for example, to improve visibility and efficiency. However, the placement of additional working ports may contribute to post-operative pain and increases risks, such as additional bleeding and adjacent organ damage.

The following published patent applications include information that may be useful in understanding the present medical devices, systems, and methods: (1) International Application No. PCT/US2009/063987, filed on Nov. 11, 2009, and published as WO 2010/056716; (2) U.S. patent application Ser. No. 10/024,636, filed Dec. 14, 2001, and published as Pub. No. US 2003/0114731; (3) U.S. patent application Ser. No. 10/999,396, filed Nov. 30, 2004, published as Pub. No. US 2005/0165449, and issued as U.S. Pat. No. 7,429,259; (4) U.S. patent application Ser. No. 11/741,731, filed Apr. 28, 2007, published as Pub. No. US 2007/0255273 and issued as U.S. Pat. No. 7,691,103; (5) U.S. patent application Ser. No. 12/146,953, filed Jun. 26, 2008, and published as Pub. No. US 2008/0269779; (6) International Patent Application No. PCT/US10/21292, filed Jan. 16, 2010, and published as WO 2010/083480.

### SUMMARY

This disclosure includes embodiments of apparatuses, systems, and methods.

Some embodiments of the present apparatuses (e.g., for use with a magnetic platform configured to be magnetically coupled to a medical device disposed within a body cavity of a patient through a tissue) comprise: a spacer having a coupling portion and a ridge defining an interior region, the coupling portion configured to be coupled to the magnetic platform such that a user can grasp the magnetic platform with at least a part of the user's hand disposed in the interior region. In some embodiments, the ridge of the spacer has an outer surface that defines an outer perimeter of the spacer, and an inner surface that defines the interior region. In some embodiments, the spacer includes a bottom surface configured to contact a surface on which the magnetic platform is disposed if the spacer is coupled to the magnetic platform. In some embodiments, the bottom surface is substantially planar. In some embodiments, the bottom surface is curved. In some embodiments, the spacer includes a curved surface between the bottom surface and the outer surface. In some embodiments, the ridge has a top end and a bottom end, and the cross-sectional thickness between the inner surface and the outer surface is larger at the bottom end than at the top end. In some embodiments, the ridge has a triangular cross-sectional shape. In some embodiments, the ridge includes one or more projections extending from the outer surface and away from the interior region. In some embodiments, the one or more projections includes a single projection extending around the outer perimeter of the spacer. In some embodiments, the coupling portion is configured to be coupled to the magnetic platform such that the strength of the magnetic field at the outermost point on the outer surface of the ridge is less than half of the strength of the strongest magnetic field immediately adjacent to the magnetic platform. In some embodiments, the coupling portion is configured to be coupled to the magnetic platform such that the strength of the magnetic field at the outermost point on the outer surface of the ridge is less than 150 Gauss.

In some embodiments of the present apparatuses, the ridge has a top end and a bottom end, and the cross-sectional thickness between the inner surface and the outer surface is substantially constant along a majority of a height of the ridge between the bottom end and the top end. In some embodiments, the ridge has a rectangular cross-sectional shape. In some embodiments, the ridge is circular. In some embodiments, the ridge has an outer transverse dimension of at least 6 inches. In some embodiments, the coupling portion is configured to be coupled to the magnetic platform such that the magnetic platform is tiltable relative to the spacer and at least a portion of the platform is substantially fixed laterally relative to the spacer. In some embodiments, the coupling portion of the spacer is coupled to a magnetic platform that is configured to be magnetically coupled to a medical device disposed within a body cavity of a patient through a tissue. Some embodiments further comprise: a second identical spacer coupled to a magnetic platform that is configured to be magnetically coupled to a medical device disposed within a body cavity of a patient through a tissue; where the magnetic platforms and spacers are configured such that if placed on a surface with the outer surfaces of the spacers in contact, the attractive force between the magnetic platforms will not exceed 2000 grams.

Some embodiments of the present apparatuses (e.g., for transport and/or storage of a magnetic platform configured to be magnetically coupled to a medical device disposed within a body cavity of a patient through a tissue) comprise: a sidewall configured to define an interior region sized to receive a magnetic platform that is configured to be magnetically coupled to a medical device disposed within a body cavity of a patient through a tissue; and a plurality of projections extending into the interior region and configured to hold a magnetic platform in the interior region in a substantially fixed orientation relative to the sidewall. In some embodiments, the sidewall comprises a plurality of openings extending through the sidewall. In some embodiments, the plurality of projections are configured to hold a magnetic platform received in the interior region such that the magnetic platform is spaced apart from the sidewall. In some embodiments, the plurality of projections are configured to hold the magnetic platform such that the strength of the magnetic field immediately outside a non-horizontal portion of the sidewall is less than 200 Gauss. In some embodiments, a magnetic platform is received in the interior region. Some embodiments further comprise: a second identical apparatus in which a second identical magnetic platform is received; where the magnetic platforms and apparatuses are configured such that if the apparatuses are placed on a horizontal planar surface with their sidewalls in contact, the attractive force between the magnetic platforms will not exceed 2000 grams. In some embodiments, the interior region is rectangular. In some embodiments, the apparatus further comprises: a first member comprising a first portion of the sidewall; and a second member comprising a second portion of the sidewall; where the first and second members are coupled together to be movable between: (i) a closed configuration in which the first and second members cooperate to define the interior region; and (ii) an open configuration in which a magnetic platform can be inserted into or removed from the interior region. In some embodiments, the first member is movably coupled to the second member by a hinge. In some embodiments, the first member is unitary with the second member. In some embodiments, at least one of the plurality of protrusions is coupled to the first member, and at least one of the plurality of protrusions is coupled to the second member. In some embodiments, the at least one protrusion coupled to the first member comprises a first plurality of ribs, and the at least one protrusion coupled to the second member comprises a second plurality of ribs. In some embodiments, the first plurality of ribs and second plurality of ribs are configured to hold a magnetic platform in a substantially fixed position relative to the sidewall if the first and second members are in the closed configuration. In some embodiments, the first plurality of ribs is unitary with the first member, and the second plurality of ribs is unitary with the second member.

In some embodiments of the present apparatuses, the interior region is circular. In some embodiments, the sidewall defines a cylinder. Some embodiments further comprise: one or more supports coupled to the sidewall and configured to support the magnetic platform in the interior region. Some embodiments further comprise: a lid configured to be coupled to sidewall; and one or more projections coupled to the lid and configured to extend into the interior region if the lid is coupled to the sidewall; where the apparatus is configured to substantially fix the position of a magnetic platform received in the interior region between the one or more projections coupled to the lid and the one or more supports coupled to the sidewall. In some embodiments, at least one of the one or more projections coupled to the lid comprises a tip biased in a direction that extends into the interior region if the lid is coupled to the sidewall.

Any embodiment of any of the present apparatuses, systems, and methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

Details associated with the embodiments described above and others are presented below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale (unless otherwise noted), meaning the sizes of the depicted elements are accurate relative to each other for at least the embodiment depicted in the figures.

FIG. 1 depicts a graphical representation of one of the present medical devices positioned within a body cavity of a patient and magnetically coupled to a positioning apparatus that is located outside the cavity.

FIG. 2 is an end view of the medical device and positioning apparatus shown in FIG. 1.

FIGS. 3A-3B depict a bottom view and a side cross-sectional view, respectively, respectively, of an embodiment of the positioning apparatus shown in FIG. 1.

FIG. 4 depicts a perspective view of an alternate positioning apparatus.

FIG. 5 depicts a perspective view of a first embodiment of the present spacer apparatuses that is configured to be coupled to the positioning apparatus of FIG. 4.

FIG. 6 depicts a side view of the spacer apparatus of FIG. 5.

FIG. 7 depicts a perspective view of the spacer apparatus of FIG. 5 coupled to the positioning apparatus of FIG. 4.

FIGS. 8A and 8B depict perspective and side views, respectively, of a second embodiment of the present spacer apparatuses.

FIGS. 9 and 10 depict perspective views of a first embodiment of the present enclosure apparatuses in closed and open configurations, respectively.

FIGS. 10-14 depict various views of a second embodiment of the present enclosure apparatuses.

### DESCRIPTION OF ILLUSTRATIVE EMBODIMENTS

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a device or kit that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps.

Further, a device or system that is configured in a certain way is configured in at least that way, but it can also be configured in other ways than those specifically described.

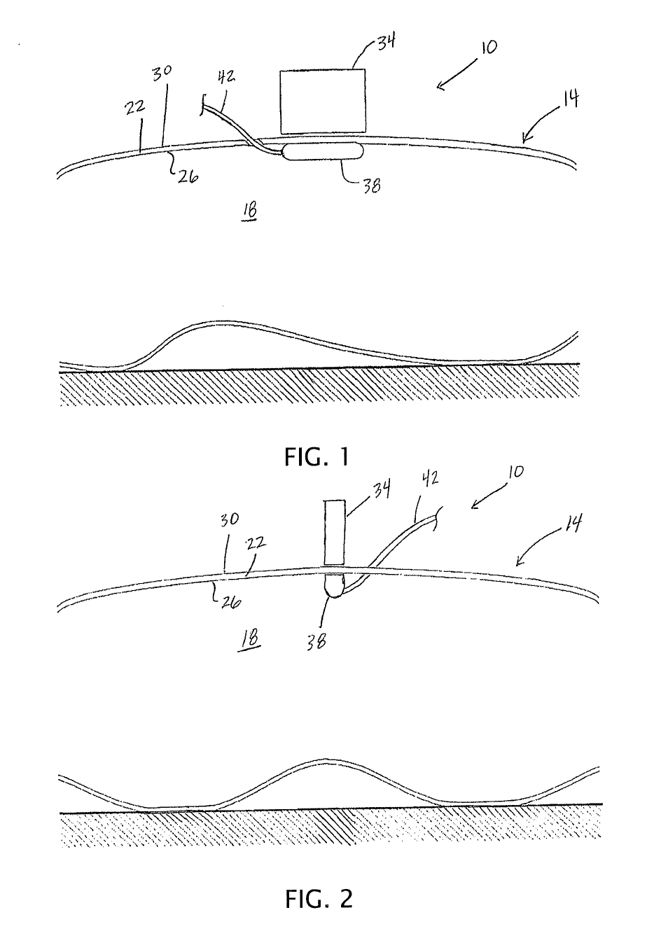

Referring now to the drawings, shown in FIGS. 1 and 2 by reference numeral 10 is one embodiment of a system for medical procedures that can be used with the present invention. System 10 is shown in conjunction with a patient 14, and more particularly in FIG. 1 is shown relative to a longitudinal cross-sectional view of the ventral cavity 18 of a human patient 14, and in FIG. 2 is shown relative to a transverse cross-sectional view of the ventral cavity of the patient. For brevity, cavity 18 is shown in simplified conceptual form without organs and the like. Cavity 18 is at least partially defined by wall 22, such as the abdominal wall, that includes an interior surface 26 and an exterior surface 30. The exterior surface 30 of wall 22 can also be an exterior surface 30 of the patient 14. Although patient 14 is shown as human in FIGS. 1 and 2, various embodiments of the present invention (including the version of system 10 shown in FIGS. 1 and 2) can also be used with other animals, such as in veterinary medical procedures.

Further, although system 10 is depicted relative to ventral cavity 18, system 10 and various other embodiments of the present invention can be utilized in other body cavities of a patient, human or animal, such as, for example, the thoracic cavity, the abdominopelvic cavity, the abdominal cavity, the pelvic cavity, and other cavities (e.g., lumens of organs such as the stomach, colon, or bladder of a patient). In some embodiments of the present methods, and when using embodiments of the present devices and systems, a pneumoperitoneum may be created in the cavity of interest to yield a relatively-open space within the cavity.

As shown in FIGS. 1 and 2, system 10 comprises a positioning apparatus or magnetic platform 34 and a medical device 38; the apparatus is configured to magnetically position the device with a body cavity of a patient. In some embodiments, apparatus 34 can be described as an exterior apparatus and/or external unit and device 38 as an interior device and/or internal unit due the locations of their intended uses relative to patients. As shown, apparatus 34 can be positioned outside the cavity 18 near, adjacent to, and/or in contact with the exterior surface 30 of the patient 14. Device 38 is positionable (can be positioned), and is shown positioned, within the cavity 18 of the patient 14 and near, adjacent to, and/or in contact with the interior surface 26 of wall 22. Device 38 can be inserted or introduced into the cavity 18 in any suitable fashion. For example, the device 18 can be inserted into the cavity through a puncture (not shown) in wall 22, through a tube or trocar (not shown) extending into the cavity 18 through a puncture or natural orifice (not shown), or may be inserted into another portion of the patient 14 and moved into the cavity 18 with apparatus 34, such as by the methods described in this disclosure. If the cavity 18 is pressurized, device 38 can be inserted or introduced into the cavity 18 before or after the cavity 18 is pressurized.

Additionally, some embodiments of system 10 include a version of device 38 that has a tether 42 coupled to and extending away from the device 38. In the depicted embodiment, tether 42 extends from device 38 and out of the cavity 18, for example, through the opening (not shown) through which device 38 is introduced into the cavity 18. The tether 42 can be flexible and/or elongated. In some embodiments, the tether 42 can include one or more conduits for fluids that can be used, for example, for actuating a hydraulic cylinder or irrigating a region within the cavity 18. In some embodiments, the tether 42 can include one or more conductors for enabling electrical communication with the device 38. In some embodiments, the tether 42 can include one or more conduits for fluid and one or more conductors. In some embodiments, the tether does not include a conduit or conductor and, instead, includes a cord for positioning, moving, or removing device 38 from the cavity 18. The tether 14, for example, can be used to assist in positioning the device 34 while the device 34 is magnetically coupled to the apparatus 38, or to remove the device 34 from the cavity 18 when device 38 is not magnetically coupled to apparatus 34.

As is discussed in more detail below, apparatus 34 and device 38 can be configured to be magnetically couplable to one another such that device 38 can be positioned or moved within the cavity 18 by positioning or moving apparatus 34 outside the cavity 18. “Magnetically couplable” means capable of magnetically interacting so as to achieve a physical result without a direct physical connection. Examples of physical results are causing device 38 to move within the cavity 18 by moving apparatus 34 outside the cavity 18, and causing device 38 to remain in a position within the cavity 18 or in contact with the interior surface 26 of wall 22 by holding apparatus 34 in a corresponding position outside the cavity 18 or in contact with the exterior surface 30 of wall 22. Magnetic coupling can be achieved by configuring apparatus 34 and device 38 to cause a sufficient magnetic attractive force between them. For example, apparatus 34 can comprise one or more magnets (e.g., permanent magnets, electromagnets, or the like) and device 38 can comprise a ferromagnetic material. In some embodiments, apparatus 34 can comprise one or more magnets, and device 38 can comprise a ferromagnetic material, such that apparatus 34 attracts device 38 and device 38 is attracted to apparatus 34. In other embodiments, both apparatus 34 and device 38 can comprise one or more magnets such that apparatus 34 and device 38 attract each other.

The configuration of apparatus 34 and device 38 to cause a sufficient magnetic attractive force between them can be a configuration that results in a magnetic attractive force that is large or strong enough to compensate for a variety of other factors (such as the thickness of any tissue between them) or forces that may impede a desired physical result or desired function. For example, when apparatus 34 and device 38 are magnetically coupled as shown, with each contacting a respective surface 26 or 30 of wall 22, the magnetic force between them can compress wall 22 to some degree such that wall 22 exerts a spring or expansive force against apparatus 34 and device 38, and such that any movement of apparatus 34 and device 38 requires an adjacent portion of wall 22 to be similarly compressed. Apparatus 34 and device 38 can be configured to overcome such an impeding force to the movement of device 38 with apparatus 34. Another force that the magnetic attractive force between the two may have to overcome is any friction that exists between either and the surface, if any, that it contacts during a procedure (such as apparatus 34 contacting a patient's skin). Another force that the magnetic attractive force between the two may have to overcome is the force associated with the weight and/or tension of the tether 42 and/or frictional forces on the tether 42 that may resist, impede, or affect movement or positioning of device 38 using apparatus 34.

In some embodiments, device 38 can be inserted into cavity 18 through an access port having a suitable internal diameter. Such access ports includes those created using a conventional laparoscopic trocar, gel ports, those created by incision (e.g., abdominal incision), and natural orifices. Device 38 can be pushed through the access port with any elongated instrument such as, for example, a surgical instrument such as a laparoscopic grasper or a flexible endoscope.

In embodiments where the tether 42 is connectable to a power source or a hydraulic source (not shown), the tether can be connected to the power source or the hydraulic source (which may also be described as a fluid source) either before or after it is connected to device 38.

In some embodiments, when device 38 is disposed within cavity 18, device 38 can be magnetically coupled to apparatus 34. This can serve several purposes including, for example, to permit a user to move device 38 within cavity 18 by moving apparatus 34 outside cavity 18. The magnetic coupling between the two can be affected by a number of factors, including the distance between them. For example, the magnetic attractive force between device 38 and apparatus 34 increases as the distance between them decreases. As a result, in some embodiments, the magnetic coupling can be facilitated by temporarily compressing the tissue (e.g., the abdominal wall) separating them. For example, after device 38 has been inserted into cavity 18, a user (such as a surgeon) can push down on apparatus 34 (and wall 22) and into cavity 18 until apparatus 34 and device 38 magnetically couple.

In FIGS. 1 and 2, apparatus 34 and device 38 are shown at a coupling distance from one another and magnetically coupled to one another such that device 38 can be moved within the cavity 18 by moving apparatus 34 outside the outside wall 22. The “coupling distance” between two structures (e.g., apparatus 34 and device 38) is defined as a distance between the closest portions of the structures at which the magnetic attractive force between them is great enough to permit them to function as desired for a given application.

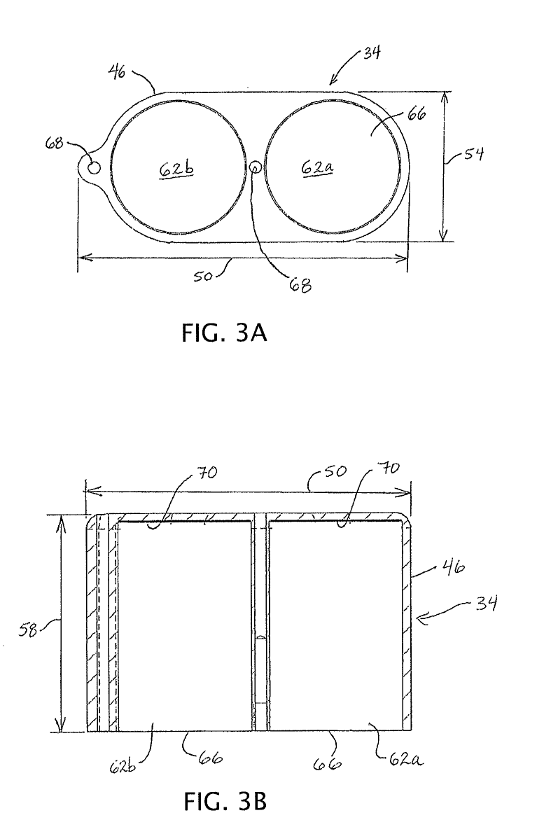

Referring now to FIGS. 3A and 3B, a bottom view and a side cross-sectional view are shown, respectively, of an embodiment of apparatus 34. Apparatus 34 has a width 50, a depth 54, and a height 58, and includes a housing 46. The apparatus (and, more specifically, housing 46) is configured to support, directly or indirectly, at least one magnetic assembly in the form of one or more magnetic field sources. In the embodiments shown, apparatus 34 is shown as including a first magnetic field source 62a and a second magnetic field source 62b. Each magnetic field source 62a, 62b has a coupling end 66 and a distal end 70. As described in more detail below, the coupling ends face device 38 when apparatus 34 and device 38 are magnetically coupled. The depicted embodiment of housing 46 of apparatus 34 also includes a pair of guide holes 68 extending through housing 46 for guiding, holding, or supporting various other devices or apparatuses, as described in more detail below. In other embodiments, the housing of apparatus 34 can have any other suitable number of guide holes 68 such as, for example, zero, one, three, four, five, or more guide holes 68. In some embodiments, housing 46 comprises a material that is minimally reactive to a magnetic field such as, for example, plastic, polymer, fiberglass, or the like. In other embodiments, housing 46 can be omitted or can be integral with the magnetic field sources such that the apparatus is, itself, a magnetic assembly comprising a magnetic field source.

Magnets, in general, have a north pole (the N pole) and a south pole (the S pole). In some embodiments, apparatus 34 can be configured (and, more specifically, its magnetic field sources can be configured) such that the coupling end 66 of each magnetic field source is the N pole and the distal end 70 of each magnetic field source is the S pole. In other embodiments, the magnetic field sources can be configured such that the coupling end 66 of each magnetic field source is the S pole and the distal end 70 of each magnetic field source is the N pole. In other embodiments, the magnetic field sources can be configured such that the coupling end of the first magnetic field source 62a is the N pole and the recessed end of the first magnetic field source 62a is the S pole, and the coupling end of the second magnetic field source 62b is the S pole and the recessed end of the second magnetic field source 62b is the N pole. In other embodiments, the magnetic field sources can be configured such that the coupling end of the first magnetic field source 62a is the S pole and its recessed end is the N pole, and the coupling end of the second magnetic field source 62b is the N pole and its recessed end is the S pole.

In the embodiment shown, each magnetic field source includes a solid cylindrical magnet having a circular cross section. In other embodiments, each magnetic field source can have any suitable cross-sectional shape such as, for example, rectangular, square, triangular, fanciful, or the like. In some embodiments, each magnetic field source comprises any of: any suitable number of magnets such as, for example, one, two, three, four, five, six, seven, eight, nine, ten, or more magnets; any suitable number of electromagnets such as, for example, one, two, three, four, five, six, seven, eight, nine, ten or more electromagnets; any suitable number of pieces of ferromagnetic material such as, for example, one, two, three, four, five, six, seven, eight, nine, ten or more pieces of ferromagnetic material; any suitable number of pieces of paramagnetic material such as, for example, one, two, three, four, five, six, seven, eight, nine, ten or more pieces of paramagnetic material; or any suitable combination of magnets, electromagnets, pieces of ferromagnetic material, and/or pieces of paramagnetic material.

In some embodiments, each magnetic field source can include four cylindrical magnets (not shown) positioned in end-to-end in linear relation to one another, with each magnet having a height of about 0.5 inch and a circular cross-section that has a diameter of about 1 inch. In these embodiments, the magnets can be arranged such that the N pole of each magnet faces the S pole of the next adjacent magnet such that the magnets are attracted to one another and not repulsed.

In some embodiments, device 38 can also include one or more magnets or other magnetically-attractive elements that can be attracted to magnetic field sources 62a and 62b to enable magnetic coupling between apparatus 34 and 38.

Examples of suitable magnets can include: flexible magnets; Ferrite, such as can comprise Barium or Strontium; AlNiCo, such as can comprise Aluminum, Nickel, and Cobalt; SmCo, such as can comprise Samarium and Cobalt and may be referred to as rare-earth magnets; and NdFeB, such as can comprise Neodymium, Iron, and Boron. In some embodiments, it can be desirable to use magnets of a specified grade, for example, grade 40, grade 50, or the like. Such suitable magnets are currently available from a number of suppliers, for example, Magnet Sales & Manufacturing Inc., 11248 Playa Court, Culver City, Calif. 90230 USA; Amazing Magnets, 3943 Irvine Blvd. #92, Irvine, Calif. 92602; and K & J Magnetics Inc., 2110 Ashton Dr. Suite 1A, Jamison, Pa. 18929. In some embodiments, one or more magnetic field sources can comprise ferrous materials (e.g., steel) and/or paramagnetic materials (e.g., aluminum, manganese, platinum).

In some embodiments, apparatus 34 and device 38 can be configured to have a minimum magnetic attractive force or “coupling force” at a certain distance. For example, in some embodiments, apparatus 34 and device 38 can be configured such that at a distance of 50 millimeters between the closest portions of apparatus 34 and device 38, the magnetic attractive force between apparatus 34 and device 38 is at least about: 20 grams, 25 grams, 30 grams, 35 grams, 40 grams, or 45 grams. In some embodiments, apparatus 34 and device 38 can be configured such that at a distance of about 30 millimeters between the closest portions of apparatus 34 and device 38, the magnetic attractive force between them is at least about: 25 grams, 30 grams, 35 grams, 40 grams, 45 grams, 50 grams, 55 grams, 60 grams, 65 grams, 70 grams, 80 grams, 90 grams, 100 grams, 120 grams, 140 grams, 160 grams, 180 grams, or 200 grams. In some embodiments, apparatus 34 and device 38 can be configured such that at a distance of about 15 millimeters between the closest portions of apparatus 34 and device 38, the magnetic attractive force between them is at least about: 200 grams, 250 grams, 300 grams, 350 grams, 400 grams, 45 grams, 500 grams, 550 grams, 600 grams, 650 grams, 700 grams, 800 grams, 900 grams, or 1000 grams. In some embodiments, apparatus 34 and device 38 can be configured such that at a distance of about 10 millimeters between the closest portions of apparatus 34 and device 38, the magnetic attractive force between them is at least about: 500 grams, 1000 grams, 2000 grams, 2200 grams, 2400 grams, 2600 grams, 2800 grams, 3000 grams, 3200 grams, 3400 grams, 3600 grams, 3800 grams, or 4000 grams.

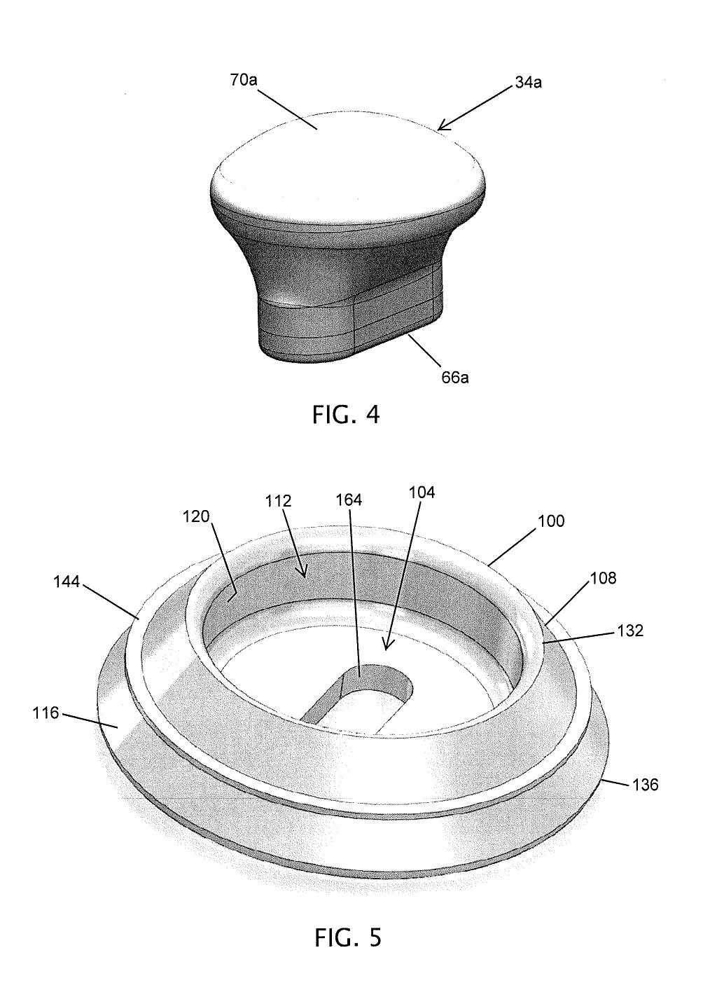

FIG. 4 depicts a perspective view of an alternate positioning apparatus or magnetic platform 34a. Apparatus 34a is substantially similar to apparatus 34, with the primary exception that housing 46a has a distal end 70a that is enlarged relative to coupling end 66a to facilitate being grasped by a user's hand.

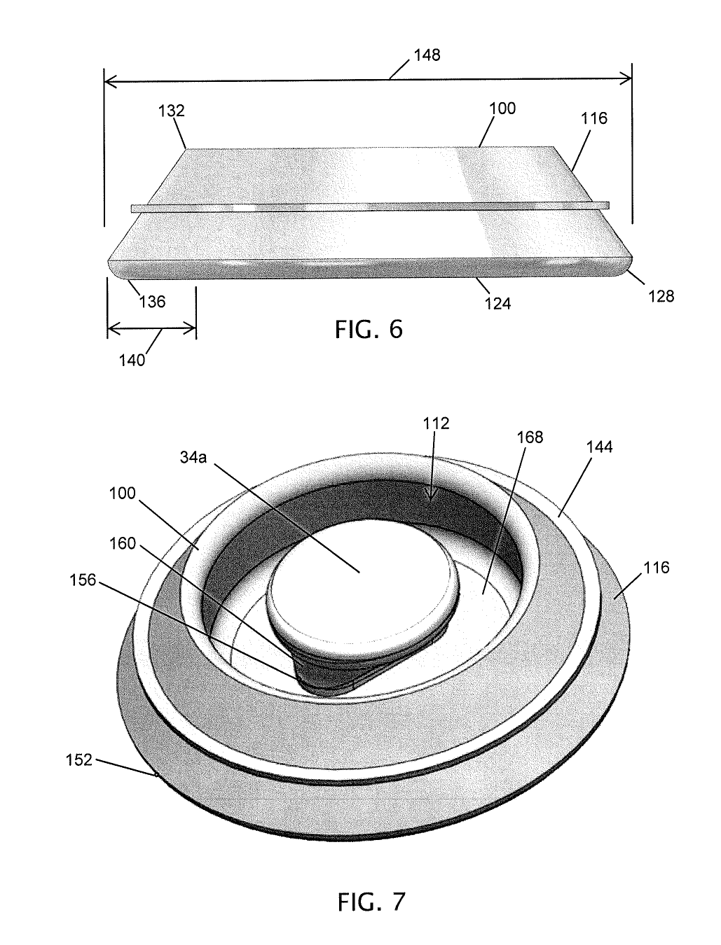

Referring now to FIGS. 5-7, FIG. 5 depicts a perspective view of a first embodiment 100 of the present spacer apparatuses that is configured to be coupled to the apparatus 34a; FIG. 6 depicts a side view of spacer 100; and FIG. 7 depicts a perspective view of spacer 100 coupled to apparatus 34a. In the embodiment shown, spacer 100 has a coupling portion 104 and a ridge 108 defining an interior region 112. Coupling portion 104 is configured to be coupled to apparatus or platform 34a (e.g., to coupling end 66a, as in the embodiment shown) such that a user can grasp apparatus 34a with at least a part of the user's hand (e.g., up to all of the user's hand) disposed in interior region 112 (e.g., such that ridge 108 may protect the user's hand from impacting adjacent apparatuses 34a and/or certain other objects to which apparatus 34a may be attracted). In the embodiment shown, ridge 108 has an outer surface 116 that defines an outer perimeter of the spacer, and an inner surface 120 that defines the interior region (e.g., defines a cylindrical interior region, as in the embodiment shown).

In the embodiment shown, spacer 100 includes a bottom surface 124 configured to contact a surface (e.g., the skin of a patient's abdomen during use, a shelf during storage and/or cleaning, and/or the like) on which a magnetic platform (e.g., apparatus 34a) is disposed if the spacer is coupled to the magnetic platform. In the embodiment shown, bottom surface 124 is substantially planar. In other embodiments, the bottom surface can be curved (e.g., concave) to facilitate sliding along a surface. For example, during laparoscopic surgery, a patient's abdomen may be pressurized and become notably convex, such that the bottom surface of the spacer can be made concave to more closely correspond to the shape of the patient's pressurized abdomen to increase the surface area of the bottom surface that contacts the patient's skin. In the embodiment shown, spacer 100 also includes a curved surface 128 (e.g., a filet) between bottom surface 124 and outer surface 116, such as, for example, to facilitate sliding relative to a patient's skin during use.

In the embodiment shown, ridge 108 is circular (when viewed from the top). In the embodiment shown, ridge 108 has a top end 132 and a bottom end 136, and the cross-sectional thickness 140 between inner surface 120 and outer surface 116 is larger at bottom end 136 than at top end 132. For example, in the embodiment shown, ridge 108 has a triangular cross-sectional shape (e.g., at any point along ridge 108, as in the embodiment shown which has a substantially constant cross-sectional shape). In some embodiments, ridge 108 includes one or more projections extending from the outer surface and away from the interior region. For example, in the embodiment shown, ridge 108 includes a single projection 144 extending from outer surface 116 and away from interior region 112 around the outer perimeter of spacer 100. In some embodiments, ridge 108 has an outer transverse dimension of at least 6 inches. For example, in the embodiment shown, ridge 108 has a diameter 148 measured to the outermost portion of outer surface 116 of at least 4 inches (e.g., equal to, or between any two of: 4, 5, 6, 7, 8, 9, 10, 11, 12, or more inches). In some embodiments, ridge 108 has a non-circular shape (e.g., ellipse, rectangular, or other shape corresponding to the outer shape of the coupling end of apparatus 34a) with a first transverse dimension (e.g., minor diameter) of at least 5 inches and a second transverse dimension (e.g., major diameter) of at least 7 inches. For example, in some embodiments, ridge 108 is configured to have an outer perimeter that is spaced from the outer perimeter of the coupling end of apparatus 34a by a distance of at least 1.5 inches (e.g., 2 inches or more).

In the embodiment shown, coupling portion 104 is configured to be coupled to apparatus 34a such that the strength of the magnetic field (of the magnetic field source(s) of apparatus 34a) at the outermost (relative to the center of interior region 112) point (e.g., point 152) on outer surface 116 of the ridge is less than half of the strength of the strongest magnetic field immediately adjacent to apparatus 34a (e.g., at a point 156 on the outer surface 160 of apparatus 34a in the same horizontal (relative to apparatus 34a) plane as the outermost point 152 of spacer 100). Stated another way, spacer 100 is configured to be coupled to apparatus 34a such that the distance between outer surface 116 of ridge 108 and outer surface 160 of apparatus 34a is large enough that, at any point on along the outermost perimeter of the spacer (e.g., the intersection of outer surface 116 and curved surface 128 in the embodiment shown), the strength of the magnetic field of apparatus 34a is less than half (e.g., equal to, or between any two of: 40%, 30%, 20%, or less) of its strongest point on surface 160. For example, in some embodiments, coupling portion 104 is configured to be coupled to apparatus 34a such that the strength of the magnetic field (of the magnetic field source(s) of apparatus 34a) at the outermost (relative to the center of interior region 112) point (e.g., point 152) on outer surface 116 of the ridge is less than 200 Guass (e.g., less than, or between any two of: 200, 150, and 100 Guass).

In the embodiment shown, coupling portion 104 is configured to be coupled to the apparatus 34a such that if the spacer and the apparatus are disposed on a horizontal planar surface such that the spacer (e.g., outer surface 116) contacts an identical second spacer (e.g., the outer surface 116 of the identical second spacer) that is coupled to an identical second magnetic platform 34a, an attractive force between the two magnetic platforms will not exceed 2000 grams. Stated another way, spacer 100 is configured (e.g., coupling portion 104 is positioned relative to outer surface 116 of ridge 108) such that if two spacers 100 are coupled to apparatuses 34a and the spacers are positioned in contact with each other, the attractive force (the force that a user must overcome to separate one apparatus/spacer assembly from the other apparatus/spacer assembly) will not exceed 2000 grams (e.g., equal to, or between any two of: 1500 grams, 1000 grams, 500 grams, or less).

Some of the present embodiments comprise two identical spacers (e.g., 100) coupled to a identical magnetic platforms (e.g., 34a); where the magnetic platforms and spacers are configured such that if placed on a surface with the outer surfaces of the spacers in contact, the attractive force between the magnetic platforms will not exceed 2000 grams.

In the embodiment shown, coupling portion 104 includes an opening 164 configured (e.g., sized and shaped) to receive coupling end 66a of apparatus 34a. In some embodiments, coupling portion 104 is configured to be coupled to apparatus 34a such that apparatus 34a is tiltable relative to spacer 100 and at least a portion of apparatus is substantially fixed laterally relative to the spacer. For example, in the embodiment shown, opening 164 is larger than coupling end 66a of apparatus 34a, and/or a bottom portion 168 of spacer 100 is flexible, such that coupling end 66a is prevented from moving laterally more than a minimal distance relative to the spacer, while still permitting distal end 70a to move relative to spacer 100 to tilt apparatus 34a. Spacer 100 can, for example, comprise a non-ferrous and/or magnetically-inert material (e.g., a polymer).

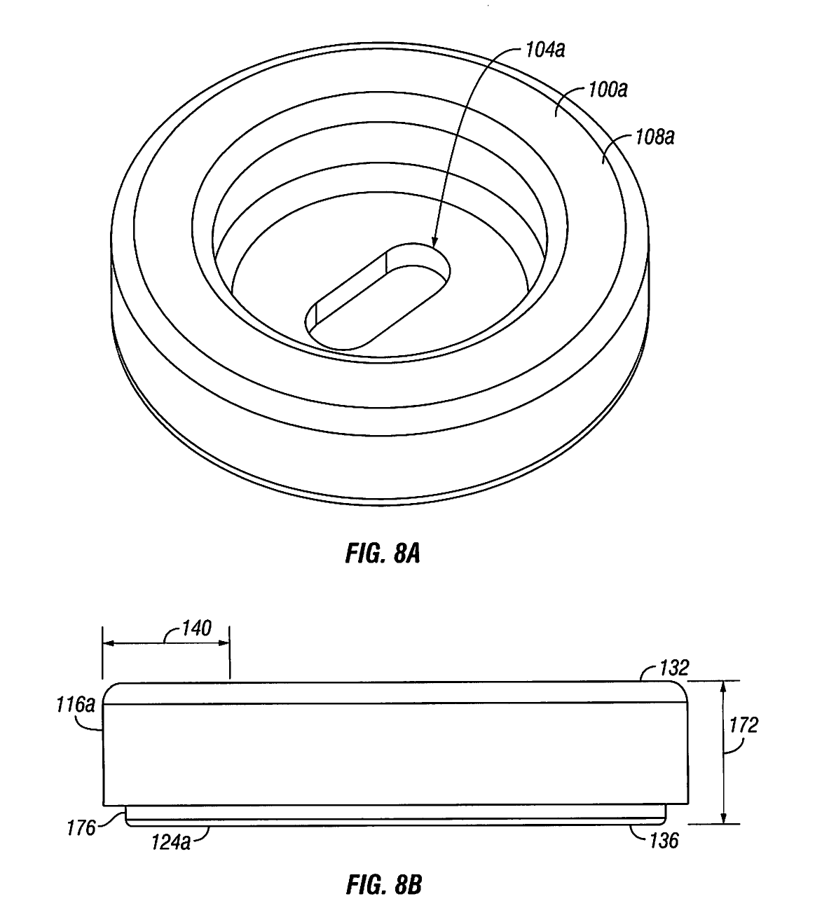

FIGS. 8A and 8B depict perspective and side views, respectively, of a second embodiment 100a of the present spacer apparatuses. Spacer 100a is substantially similar to spacer 100, with the primary exception that the cross-sectional thickness 140 is substantially constant along a majority of a height 172 of the ridge between the bottom end 136 and the top end 132 (e.g., ridge 108a has a rectangular cross-sectional shape). In the embodiment shown, spacer 100a includes a ledge 176 around the bottom end 136 of ridge 108a (between curved surface 128a and outer surface 116a).

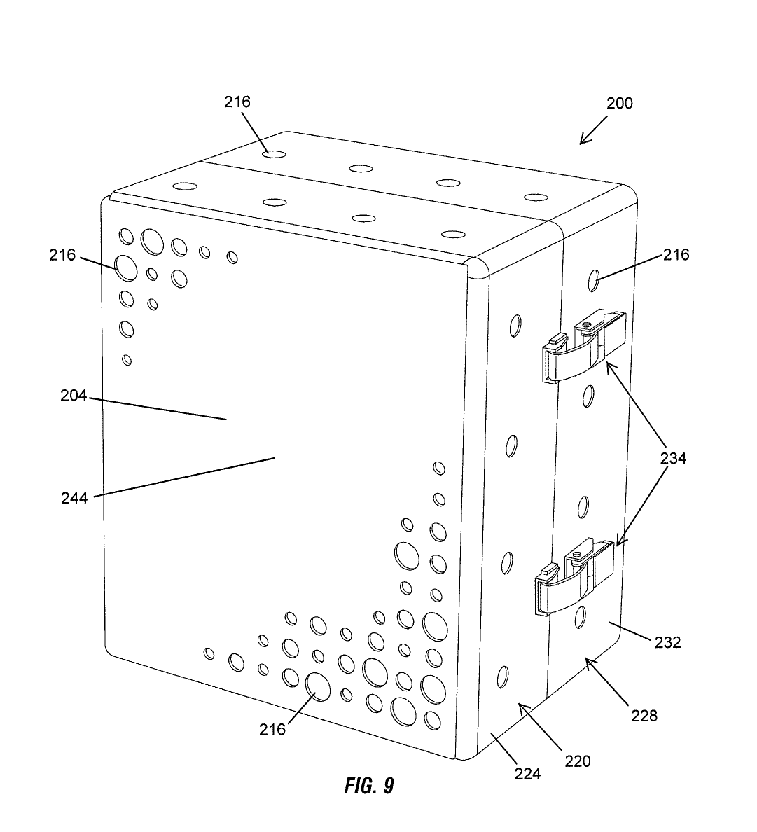

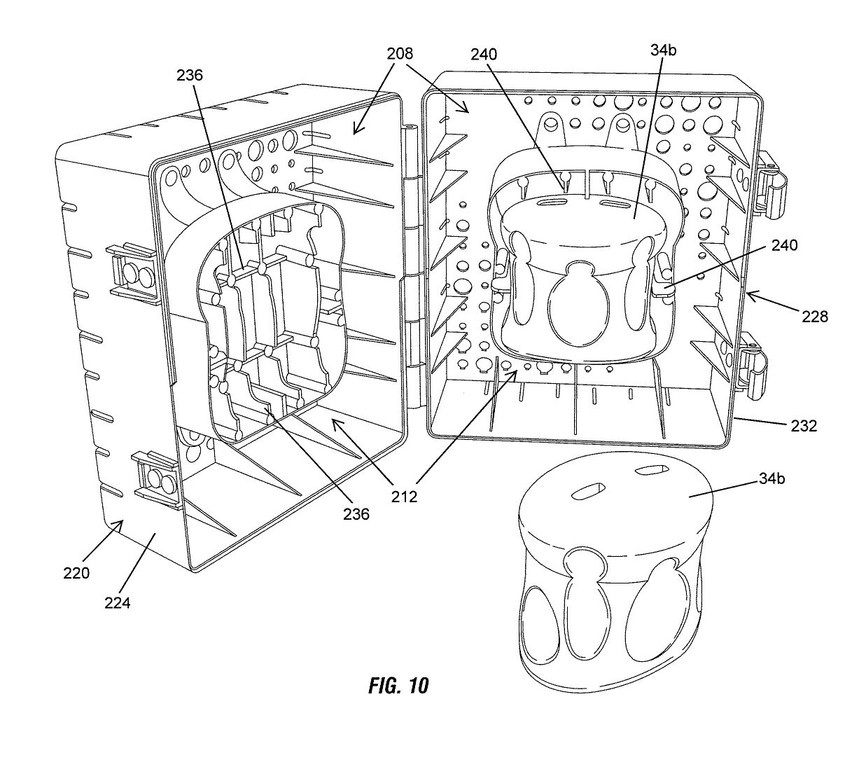

Referring now to FIGS. 9 and 10, shown therein are perspective views of a first embodiment 200 of the present enclosure apparatuses (shown in a closed configuration in FIG. 9 and an open configuration in FIG. 10). Embodiments of enclosure 200 can, for example, be used for transport, sterilization, and/or storage of magnetic platforms to prevent undesirable magnetic interactions with other magnetic platforms (e.g., to prevent to adjacent magnetic platforms from becoming magnetically attracted to one another to a degree that separation becomes difficult). In the embodiment shown, enclosure 200 comprises: a sidewall 204 configured to define an interior region 208 sized to receive a magnetic platform (apparatus 34b); and a plurality of projections 212 extending into the interior region and configured to hold a magnetic platform 34b (FIG. 10) in the interior region in a substantially fixed orientation relative to the sidewall. In this embodiment, interior region 208 is rectangular. In the embodiment shown, sidewall 204 comprises a plurality of openings 216 extending through the sidewall. Openings 216 can, for example, be used to introduce a cleaning and/or sterilizing agent (e.g., liquid) into interior region 208 to clean and/or sterilize an apparatus 34b disposed in the enclosure. In the embodiment shown, apparatus 34b is substantially similar to apparatuses 34 and 34a, with the primary difference that distal end 70b has a different shape that is enlarged relative to coupling end 66b, but not as much so as distal end 70a of apparatus 34a relative to coupling end 66a. Sidewall 204 can comprise, for example, a polymer (e.g., a substantially rigid plastic).

In the embodiment shown, projections 212 are configured to hold a magnetic platform (e.g., apparatus 34b) received in the interior region (as shown in FIG. 10) such that the magnetic platform apparatus is spaced apart from sidewall 204. In the embodiment shown, enclosure 200 comprises: a first member 220 comprising a first portion 224 of sidewall 204; and a second member 228 comprising a second portion 232 of sidewall 204. In this embodiment, first member 220 and second member 228 are coupled together to be movable between: (i) a closed configuration (FIG. 9) in which the first and second members cooperate to define the interior region; and (i) an open configuration (FIG. 10) in which a magnetic platform (e.g., apparatus 34b) can be inserted into or removed from the interior region. First and second members 220 and 228 can be coupled together by a hinge 234. In other embodiments, first member 220 can be unitary with second member 228 (e.g., coupled together by a “living hinge” unitary with first and second members 220 and 228).

In the embodiment shown, at least one protrusion 212 is coupled to first member 220, and at least one protrusion 212 is coupled to second member 228. In the embodiment shown, the at least one protrusion coupled to the first member comprises a plurality of ribs 236 (e.g., a plurality of intersecting planar ribs extending inward from first portion 224 of sidewall 204), and the at least one protrusion coupled to the second member comprises a plurality of ribs 240 (e.g., a plurality of intersecting planar ribs extending inward from second portion 232 of sidewall 204). In this embodiment, ribs 236 and ribs 240 are configured to hold a magnetic platform (e.g., apparatus 34b) in a substantially fixed position relative to the sidewall if the first and second members are in the closed configuration (FIG. 10). In the embodiment shown, ribs 236 are unitary with first member 220, and ribs 240 are unitary with second member 228. As shown, in this embodiment, ribs 236 and ribs 240 are contoured to correspond to the shape of the magnetic platform. In the embodiment shown, enclosure 200 comprises a plurality of latches 242 configured to couple first member 220 to second member 228 in the closed configuration.

In some embodiments, projections are configured to hold the magnetic platform (e.g., apparatus 34b) such that the strength of the magnetic field immediately outside a non-horizontal portion 244 of sidewall 204 is less than 300 Guass (e.g., less than 250 Gauss, less than 200 Gauss, or less). For example, in the embodiment shown, apparatus 200 is configured such that if apparatus 34b is disposed in enclosure 200, a distance of at least 3.5 centimeters (cm) separates each point on the exterior surface of apparatus 34b and the closest point on sidewall 244 (e.g., resulting in a maximum magnetic field strength immediately outside sidewall 244 of 200 Guass or less).

In some embodiments, projections 212 are configured to hold a magnetic platform (e.g., apparatus 34b) such that if the enclosure is disposed on a horizontal planar surface in contact with an identical second enclosure that is also disposed on the planar surface, and an identical second magnetic platform (e.g., apparatus 34b) is received in the second apparatus, an attractive force between the two magnetic platforms will not exceed 2000 grams (e.g., equal to, or between any two of: 1500 grams, 1000 grams, 500 grams, or less). Some of the present embodiments comprise two identical enclosures (e.g., 200) each holding a different one of two identical magnetic platforms (e.g., 34b); where the magnetic platforms and enclosures are configured such that if placed on a surface with the sidewalls of the enclosures in contact, the attractive force between the magnetic platforms will not exceed 2000 grams (e.g., equal to, or between any two of: 1500 grams, 1000 grams, 500 grams, or less).

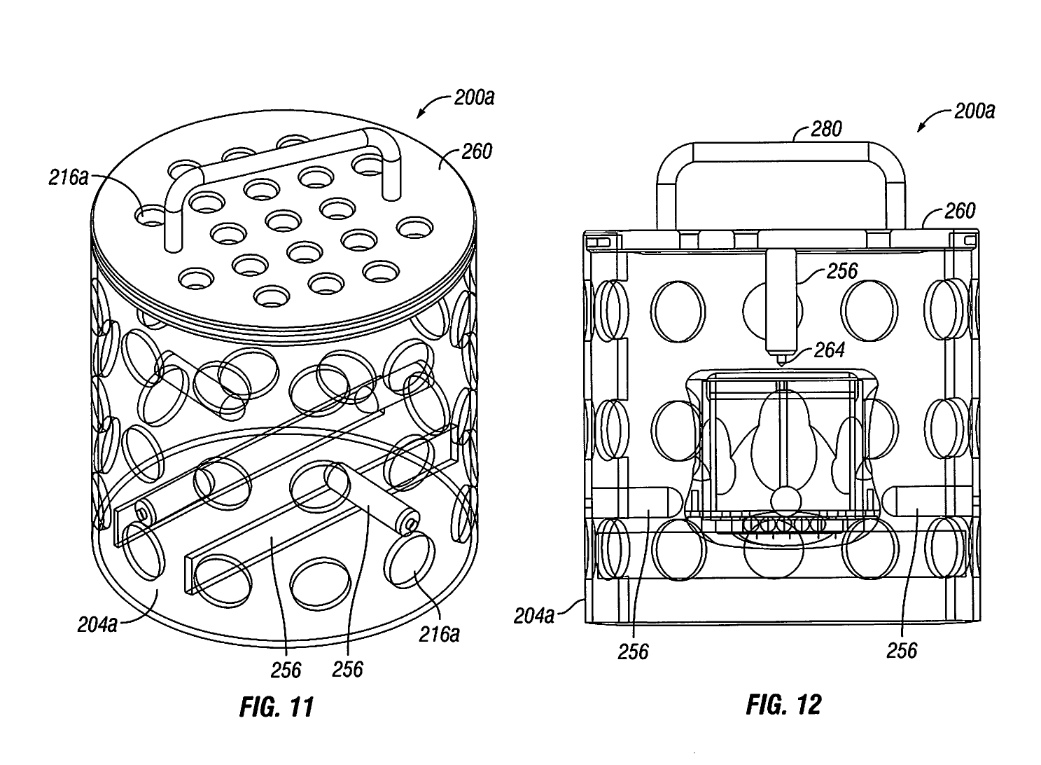

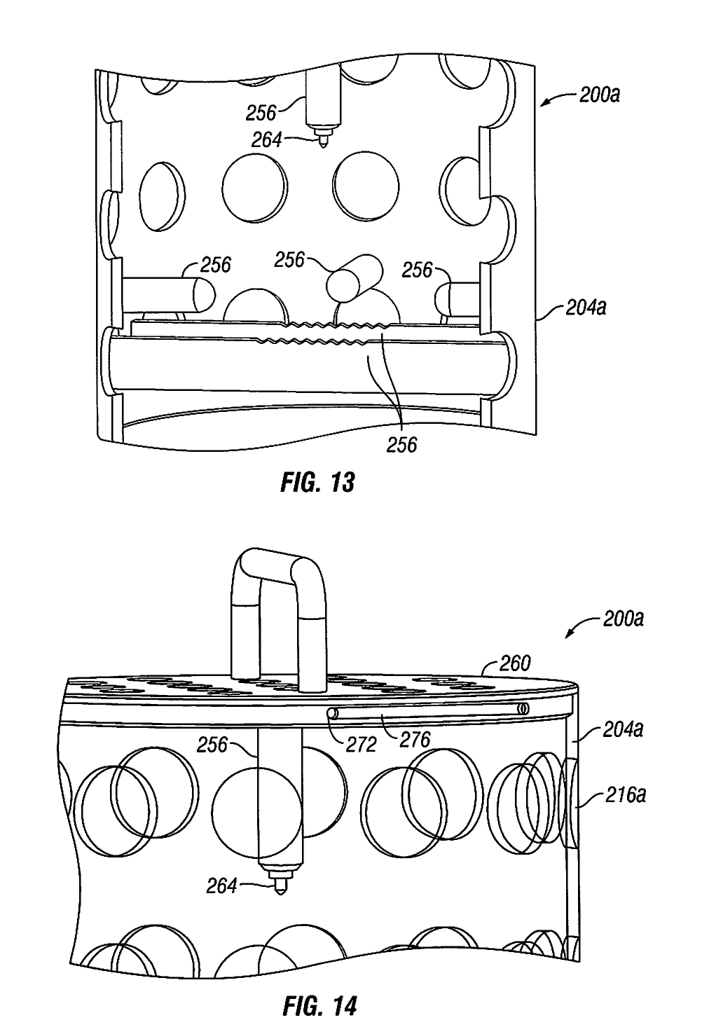

FIGS. 10-14 depict various views of a second embodiment 200a of the present enclosure apparatuses. Enclosure 200a is similar in some respects to enclosure 200, with the primary exceptions being that enclosure 200a sidewall 204a defines a cylinder having a circular interior region 208a. In some embodiments, enclosure 200a comprises one or more supports coupled to sidewall 204a and configured to support the magnetic platform (e.g., apparatus 34b) in the interior region. For example, in the embodiment shown, enclosure 200a comprises a plurality of supports 256 coupled to sidewall 204a and configured to support apparatus 34b in the interior region (e.g., in a substantially fixed orientation relative to sidewall 204a). In this embodiment, sidewall 204a and lid 260 each includes a plurality of holes 216a.

In this embodiment, apparatus 200a further comprises a lid 260 configured to be coupled to sidewall 204a, where at least one of the plurality of supports 256 is coupled to the lid and configured to extend into the interior region if the lid is coupled to the sidewall. As shown, in this embodiment, the apparatus is configured to substantially fix the position of a magnetic platform (e.g., apparatus 34b) received in the interior region between the at least one support 256 coupled to the lid and the one or more supports 256 coupled to the sidewall. In the embodiment shown, the support 256 coupled to lid 260 comprises a tip or plunger 264 biased in a direction 268 that extends into the interior region if the lid is coupled to the sidewall such that tip 264 is compressed as lid is coupled to sidewall 204a to provide downward pressure on magnetic platform 34b and thereby securely hold platform 34b in interior region. In the embodiment shown, some of supports 256 (bottom supports) extend across the entire interior region to support the magnetic platform 34b at a lower end of the interior region, some of supports 256 (side supports) are configured to extend between sidewall 204a and magnetic platform 34b, and at least one support 256 (top support) is configured to extend between lid 260 and magnetic platform 34b. In the embodiment shown, one of lid 260 and sidewall 204a includes a protrusion 272, and the other of lid 260 and sidewall 204a includes an L-shaped grooved 276, such that lid 260 can be inserted and turned relative to sidewall 204a to lock lid 260 relative to sidewall 204a. In this embodiment, lid 260 includes a handle 280 configured to be used to carry enclosure 200a when lid 260 is coupled to sidewall 204a (e.g., if a magnetic platform is in the interior region). Supports 256 can comprise, for example, aluminum and/or polymer (e.g., a substantially rigid plastic).

Embodiments of the present methods can include coupling each of one or more of the present magnetic platforms (e.g., 34, 34a, 34b) to an embodiment of the present spacers (e.g., 100, 100a), magnetically coupling the magnetic platform to a medical device (e.g., in a body cavity of a patient), disposing one or more of the present magnetic platforms (e.g., 34, 34a, 34b) in an embodiment of the present enclosures (e.g., 200, 200a). For example, multiple ones of the present magnetic platforms (34, 34a, 34b) coupled to the present spacers can be used in proximity to one another with a distance (and attractive force) between the magnetic platforms that is limited by the spacers. Similarly, multiple ones of the present magnetic platforms disposed in the present enclosures (200, 200a) can be placed adjacent to one another with a distance (and attractive force) between the magnetic platforms that is limited by the enclosures.

The above specification and examples provide a complete description of the structure and use of exemplary embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the present devices are not intended to be limited to the particular forms disclosed. Rather, they include all modifications and alternatives falling within the scope of the claims, and embodiments other than the one shown may include some or all of the features of the depicted embodiment. For example, components may be combined as a unitary structure, and/or connections may be substituted. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. An apparatus for use with a magnetic platform configured to be magnetically coupled to a medical device disposed within a body cavity of a patient through a tissue, the apparatus comprising:
a spacer having a coupling portion and a ridge defining an interior region, the coupling portion configured to be coupled to the magnetic platform such that a user can grasp the magnetic platform with at least a part of the user's hand disposed in the interior region.

2. The apparatus of claim 1, where the ridge of the spacer has an outer surface that defines an outer perimeter of the spacer, and an inner surface that defines the interior region.

3. The apparatus of claim 2, where the spacer includes a bottom surface configured to contact a surface on which the magnetic platform is disposed if the spacer is coupled to the magnetic platform.

4. The apparatus of claim 3, where the spacer includes a curved surface between the bottom surface and the outer surface.

5. The apparatus of claim 2, where the ridge includes one or more projections extending from the outer surface and away from the interior region.

6. The apparatus of claim 2, where the coupling portion is configured to be coupled to the magnetic platform such that the strength of the magnetic field at the outermost point on the outer surface of the ridge is less than half of the strength of the strongest magnetic field immediately adjacent to the magnetic platform.

7. The apparatus of claim 2, where the coupling portion is configured to be coupled to the magnetic platform such that the strength of the magnetic field at the outermost point on the outer surface of the ridge is less than 150 Gauss.

8. The apparatus of claim 1, where the ridge has an outer transverse dimension of at least 6 inches.

9. The apparatus of claim 1, where the coupling portion is configured to be coupled to the magnetic platform such that the magnetic platform is tiltable relative to the spacer and at least a portion of the platform is substantially fixed laterally relative to the spacer.

10. The apparatus of claim 1, where the coupling portion of the spacer is coupled to a magnetic platform that is configured to be magnetically coupled to a medical device disposed within a body cavity of a patient through a tissue.

11. The apparatus of claim 10, further comprising:
a second identical spacer coupled to a magnetic platform that is configured to be magnetically coupled to a medical device disposed within a body cavity of a patient through a tissue;
where the magnetic platforms and spacers are configured such that if placed on a surface with the outer surfaces of the spacers in contact, the attractive force between the magnetic platforms will not exceed 2000 grams.

12. An apparatus comprising:
a sidewall configured to define an interior region sized to receive a magnetic platform that is configured to be magnetically coupled to a medical device disposed within a body cavity of a patient through a tissue;
a plurality of projections extending into the interior region and configured to hold a magnetic platform in the interior region in a substantially fixed orientation relative to the sidewall.

13. The apparatus of claim 12, where the sidewall comprises a plurality of openings extending through the sidewall.

14. The apparatus of claim 12, where the plurality of projections are configured to hold a magnetic platform received in the interior region such that the magnetic platform is spaced apart from the sidewall.

15. The apparatus of claim 14, where the plurality of projections are configured to hold the magnetic platform such that the strength of the magnetic field immediately outside a non-horizontal portion of the sidewall is less than 200 Gauss.

16. The apparatus of claim 15, where a magnetic platform is received in the interior region.

17. The apparatus of claim 16, further comprising:
a second identical apparatus in which a second identical magnetic platform is received;
where the magnetic platforms and apparatuses are configured such that if the apparatuses are placed on a horizontal planar surface with their sidewalls in contact, the attractive force between the magnetic platforms will not exceed 2000 grams.

18. The apparatus of claim 12, where the apparatus comprises:
a first member comprising a first portion of the sidewall; and
a second member comprising a second portion of the sidewall;
where the first and second members are coupled together to be movable between:
a closed configuration in which the first and second members cooperate to define the interior region; and
an open configuration in which a magnetic platform can be inserted into or removed from the interior region.

19. The apparatus of claim 18, where at least one of the plurality of protrusions is coupled to the first member and comprises a first plurality of ribs, and at least one of the plurality of protrusions is coupled to the second member and comprises a second plurality of ribs.

20. The apparatus of claim 19, where the first plurality of ribs and second plurality of ribs are configured to hold a magnetic platform in a substantially fixed position relative to the sidewall if the first and second members are in the closed configuration.
